(19) United States
(12) Patent Application Publication
(10) Pub. No.: US 2018/0236188 A1
(43) Pub. Date: Aug. 23, 2018

(54) BLISTER FOR INHALABLE FORMULATION OF TIOTROPIUM BROMIDE

(71) Applicant: ARVEN ILAC SANAYI VE TICARET A.S., Istanbul (TR)

(72) Inventors: Ali TÜRKYILMAZ, Istanbul (TR); Devrim CELIK, Istanbul (TR)

(21) Appl. No.: 15/770,278

(22) PCT Filed: Oct. 21, 2016
(86) PCT No.: PCT/EP2016/075341

§ 371 Date: Apr. 23, 2018

(30) Foreign Application Priority Data
Oct. 23, 2015 (TR) 2015/13285

(51) Int. Cl. A61M 15/00; B65D 75/32; A61M 16/14

(52) U.S. Cl. CPC A61M 15/0051; A61M 2202/064; A61M 16/14; B65D 75/325

(57) ABSTRACT

This invention is about a blister which is an integral component of a dry powder inhaler and is structured from a base sheet and a lid sheet comprising at least two layers made from polymeric materials and flat aluminium foil, wherein the cavity that is used for storing the inhalable formulation is in the volume range of 5 to 19.8 mm³ and the cavity is filled up to 25-100%.

### FIELD OF INVENTION

This invention is about a blister which is an integral component of a dry powder inhaler is structured from a base sheet and a lid sheet comprising at least two layers made from polymeric materials and formed or flat aluminium folio, wherein cavity that is used for storing inhalable formulation is in the volume range of 5 to 19.8 mm³ and the cavity is filled up to 25-100%.

### BACKGROUND OF INVENTION

Asthma and chronic obstructive pulmonary disease (COPD) affect more than 30 million people in the United States. More than 100,000 deaths each year are attributable to these conditions. Obstruction to airflow through the lungs is the characteristic feature in each of these airway diseases, and the medications utilized in treatment are often similar. Chronic obstructive pulmonary disease (COPD) is a widespread chronic lung disorder encompassing chronic bronchitis and emphysema. The causes of COPD are not fully understood. Experience shows that the most important cause of chronic bronchitis and emphysema is cigarette smoking. Air pollution and occupational exposures may also play a role, especially when combined with cigarette smoking. Heredity also causes some emphysema cases, due to alphal anti-trypsin deficiency.

Administration of asthma drugs by an oral inhalation route is very much in focus today, because of advantages offered like rapid and predictable onset of action, cost effectiveness and high level of comfort for the user. Dry powder inhalers (DPI) are especially interesting as an administration tool, compared to other inhalers, because of the flexibility they offer in terms of nominal dose range, i.e. the amount of active substance that can be administered in a single inhalation.

Dry powder inhalers (DPI) are becoming more and more popular because of their ease of use and medical efficacy. DPI's can be divided into two major categories: bulk and pre-metered devices. Pre-metered devices are gaining more and more market share due to the ability to control the product and process of metering a correct dose to the user. DPIs with pre-metered doses are, because of this, more reliable than bulk inhalers that meter the powder dose inside the inhaler. A pre-metered DPI moves the critical step of metering a dose to a pharmaceutical production process.

Anticholinergic agents, e.g. tiotropium, especially tiotropium bromide, are effective bronchodilators. These medicaments have a relatively fast onset and long duration of action, especially tiotropium bromide, which may be active for up to 24 hours. Anticholinergic agents reduce vagal cholinergic tone of the smooth muscle, which is the main reversible component of COPD. Anticholinergic agents have been shown to cause quite insignificant side effects in clinical testing, dryness of mouth and constipation are perhaps the most common symptoms. Because it is often very difficult to diagnose asthma and COPD correctly and since both disorders may co-exist, it is advantageous to treat patients suffering temporary or continuous bronchial obstruction resulting in dyspnoea with a small but efficient dose of a long-acting anticholinergic agent, preferably tiotropium bromide, because of the small adverse side effects.

Tiotropium bromide is known from European Patent Application EP 418 716 A1 and has the following chemical structure shown as Formula I. Tiotropium bromide is the preferred anticholinergic agent because of its high potency and long duration. However, tiotropium is difficult to formulate in dry powder form to provide acceptable performance in terms of dose efficacy using prior art DPIs. Dose efficacy depends to a great deal on delivering a stable and high fine particle dose (FPD) out of the dry powder inhaler. It is also important to keep the dosage to the user as exact as possible and to maintain a stable efficacy over time, and that the medicament dose does not deteriorate during normal storage.

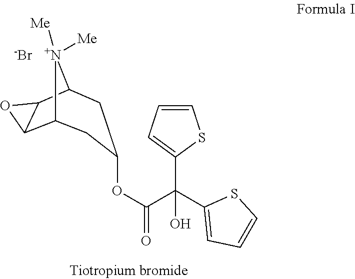

Tiotropium in pure form is a potent drug and it is therefore diluted before a dose forming step by mixing with acceptable excipients, in selected ratio (s) in order to fit a preferred method of dose forming and loading. For example, details about inhalation powders containing tiotropium in mixtures with excipients, methods of powder manufacture, use of powder and capsules for powder may be studied in the international publication WO 02/30389 Al, Bechtold-Peters et al. Manufacturing a formulation of a very small amount of e.g. tiotropium with a much larger quantity of excipient requires special precautions to be taken to give a final, stable and robust manufacturing method.

Excipients are particularly necessary in powders for inhalation if the efficacy of the pharmaceutical substance used is very high like tiotropium, so that only small amounts are needed for each individual dose. Preparing a formulation of tiotropium and an excipient where the amount of tiotropium is very small (e.g., <1:100 the amount of the excipient). In this case it is advisable to dilute the active substance so as to achieve good accuracy of flowability and metering. Additionally, maintaining the stability and preventing the agglomeration of dry powder formulation which is contained in a blister strip that is prepared for use in a single dose dry powder inhaler in order to achieve an effective inhalation should be considered.

One of the disadvantages of that methods is large amounts of excipient must be added to the formulation, to achieve the desired flowability. Because of the large amount of excipient, the properties of the inhalable powder are critically influenced by the choice of excipient. Further, the correct metered dose loaded into an inhaler to be used for the purpose of administration must be adjusted for predicted losses such as retention and more or less efficient de-aggregation of the inhaled dose.

Tiotropium is known in the industry to have problems maintaining in-use stability due to sensitivity to moisture. Conventional packaging into gelatin capsules or blister show poor performance and have a short in-use stability.

There is still need for improvement to dilute potent tiotropium and to make flowability of dry powder formulation acceptable for the dose forming process and optimize the delivered dose and maintain stable efficacy.

### DESCRIPTION OF THE INVENTION

This invention is about a blister which is an integral component of a dry powder inhaler is structured from a base sheet and a lid sheet comprising at least two layers made from polymeric materials and formed or flat aluminium folio, wherein cavity that is used for storing inhalable formulation is in the volume range of 5 to 19.8 mm³ and the cavity is filled up to 25-100%.

In a preferred embodiment, wherein the cavity is preferably in the volume range of 10 to 19.8 mm³, more preferably in the range of 15 to 19.7 mm³.

In another preferred embodiment, the cavity having the volume described above is filled preferably up to 30-100%, more preferably up to 30-70%, the most preferably up to 40-60%.

In this present invention, blister cavity is decreased and cavity filling percentage is increased. As a result of this, there is less space in every blister cavity and tiotropium exposes to air less. The inhalable powder is protected from moisture in this smaller cavities, so stability is improved.

In one embodiment, the cavity that is used for storing inhalable formulation is filled about 3-6 mg of the formulation.

According to this embodiment, the blister is in a strip form. And it comprises two sheets. The lid sheet and the base sheet of the blister strip, consists of at least two layers such as polymeric layer, aluminium foil.

According to the present invention, the polymeric materials used for making the polymeric layers which are present in both the base sheet and lid sheet are selected from the group consisting of polyethylene, polypropylene, polystyrene, polyolefin, polyamide, polyvinyl chloride, polyurethane, vinyl acetate, polybutylmethacrylate, copolymer of vinyl chloride, polyethylene terephthalate (PET), polyvinyl chloride (PVC), ortho-phthalic aldehyde (OPA) or mixtures thereof.

According to the invention, aluminium foil is used both in the lid sheet and in the base sheet of the blister strip to provide high humidity and gas protection. These layers must have the sufficient thickness which provides the protection for the stability of humidity sensitive dry powder formulation which is stored in blister cavity.

According to the embodiment, aluminium foil that is used in the lid sheet of the blister strip is in the thickness range of 5 to 40 μm, preferably of 10 to 30 μm.

In another embodiment, aluminium foil that is used in the base sheet of the blister strip is in the thickness range of 30 to 60 μm, preferably of 35 to 55 μm.

The polymeric layers which are contained in the lid sheet and the base sheet of the blister strip in accordance with the present invention may be made from either same of different polymers. The thickness of these polymeric layers depends on the type of polymeric substance used and its properties.

According to the embodiment, the polymeric layer which is used in the lid sheet of the blister strip is in the thickness range of 10 to 50 μm, preferably of 15 to 35 μm depending on the type of polymer used. The polymeric material used for making the polymeric layer is preferably polyethylene terephthalate (PET).

In another embodiment, the polymeric layer which is used in the base sheet of the blister strip is in the thickness range of 10 to 40 μm, preferably of 15 to 35 μm depending on the type of polymer used. The polymeric material used for making the polymeric layer is preferably ortho-phthalic aldehyde (OPA).

In one embodiment, the another polymeric layer which is used in the base sheet of the blister strip is in the thickness range of 80 to 120 μm, preferably of 90 to 110 μm depending on the type of polymer used. The polymeric material used for making the polymeric layer is preferably polyvinyl chloride (PVC).

Moreover, blisters which placed in the blister strips, can be in any shape wherein the polymeric layer forms a cavity molded.

The inside layer of blister cavity of said blister strip which is in contact with dry powder formulation is polymeric layer because of the fact that aluminium foil causes adhesion of part of dry powder formulation to inside layer of the cavity due to electrostatic forces, and hence cause uncontrolled dosing.

In one aspect, the present invention provides the dry powder formulation which contain: tiotropium and is inhaled from the blisters. In another aspect, the present invention provides the dry powder formulation which contains tiotropium and inhaled by providing controlled dosing of the dry powder formulation in an effective and stable way.

In another aspect, the present invention provides the dry powder formulation which contains tiotropium and is inhaled in an effective, hygienic, user-friendly way.

According to this invention, a blister which is an integral component of a dry powder inhaler wherein contains an inhalable formulation comprising tiotropium or an acceptable salt, solvate, hydrate thereof and at least one pharmaceutically acceptable excipient.

According to the invention, inhalable formulation comprising tiotropium or an acceptable salt, solvate, hydrate is tiotropium bromide.

In a preferred embodiment, the blister wherein contains an inhalable formulation comprising tiotropium bromide and at least one pharmaceutically acceptable excipient. According to the invention, said dry powder formulation comprising tiotropium bromide in an amount in the range of 0.02 to 0.03 mg.

According to this embodiment, the amount of tiotropium bromide is present in an amount of 0.25 to 0.75% by weight of total composition; preferably it is 0.35 to 0.45% by weight of total composition.

With active substances which have a particularly high efficacy, only small amounts of the active substance are needed per single dose to achieve the desired therapeutic effect. In such cases, the active substance has to be diluted with suitable excipients in order to prepare the inhalable powder. Because of the large amount of excipient, the properties of the inhalable powder are critically influenced by the choice of excipient. However, good flow properties are a prerequisite for highly accurate metering when packing and dividing up the individual doses of preparation, e.g. when producing capsules for powder inhalation or when the patient is metering the individual dose before using a multi-dose inhaler. The term inhalable proportion of active substance refers to the particles of the inhalable powder which are conveyed deep into the branches of the lungs when inhaled with a breath.

According to this invention, inhalable formulation comprising tiotropium includes at least one pharmaceutically acceptable excipient is selected from the group consisting of monosaccarides, disaccarides, polylactides, oligo- and polysaccarides, polyalcohols, glucose, arabinose, lactose, lactose monohydrate, lactose anhydrous, saccharose, maltose, dextrane, sorbitol, mannitol, xylitol, sodium chloride, calcium carbonate or mixtures thereof.

A particular excipient is lactose. In our findings regarding the sensitivity to moisture for tiotropium powders the moisture properties of any proposed excipient must be appropriate before it is selected for inclusion in a formulation comprising tiotropium, regardless of the function of the proposed excipient. An excipient which, after dose forming, gives off much water inside the container enclosing the dose of mixed powders may negatively affect the included active powder, such that the resulting fine particle dose (FPD) deteriorates rapidly after dose forming.

Therefore, excipients to be mixed with tiotropium must be selected primarily among acceptable excipients, which have good moisture qualities in the sense that the substance will not adversely affect the active medicament fine particle dose (FPD) for the shelf life of the product regardless of normal changes in ambient conditions during storage. Suitable “dry” excipients include those in the above-mentioned groups.

In a preferred embodiment at least one pharmaceutically acceptable excipient is lactose monohydrate. Lactose is selected as the dry excipient and the excipient is preferably lactose monohydrate to be used in a mixture with tiotropium. One reason for selecting lactose as excipient is its inherent property of having a low and constant water sorption isotherm.

Excipients having a similar or lower sorption isotherm may also be considered for use, provided other required qualities are met.

Ambient conditions during dose forming, loading and container sealing should be closely controlled. The temperature should preferably be below 25° C. and relative humidity should preferably be below 35% Rh. The powder formulation should also be kept as dry as possible during the dose forming process. Taking these precautions will ensure that only a very small, acceptable amount of water is enclosed in the container together with the dose and not enough to present a threat to the stability of the moisture sensitive substance and the fine particle dose (FPD). The original fine particle fraction (FPF) of the medicament dose (e.g. tiotropium) manifested in a high fine particle dose (FPD) of the metered dose of the medical product at the packaging stage is preserved in the high barrier seal container. Thus, when the pre-metered dose is delivered by a dry powder inhaler (DPI) it is unaffected for the shelf life of the medical product by normal variations in ambient conditions during handling, storage and delivery.

In order to develop a formulation of tiotropium having controlled moisture properties a study into the chemical and physical properties of the chosen excipient should first be carried out. The sorption isotherm properties will give information with respect to how a formulation will respond to different temperatures and relative humidity in its surrounding environment. One very important question is also the “memory” of some excipients built in by the fact that it takes a very long time to reach steady state for the excipient after a disturbance in the environment. A suitable excipient for a formulation comprising tiotropium is an excipient like lactose monohydrate. The isotherm of lactose monohydrate has three important properties:
Low absolute water content
Low change in absolute water content after a change in relative humidity.
Highly stable in in-use temperature situations

Low absolute water content ensures that a disturbance from steady conditions will not have a big impact on a tiotropium dose when the total amount of water present in the excipient is low. The low change in absolute water content at different relative humidity ensures that the excipient has no “memory” and that it can easily be put into a steady state at a given relative humidity before filling into a high barrier container. The temperature stability ensures that adsorption and desorption inside the high barrier seal will influence the pharmacologically active ingredient (API) as little as possible.

According to this embodiment, the amount of lactose monohydrate is present in an amount of 98.00 to 99.99% by weight of total composition; preferably it is 99.00 to 99.99% by weight of total composition.

According to the invention, said dry powder formulation comprising lactose monohydrate in an amount in the range of 2.9 to 5.9 mg, preferably in an amount in the range of 5.0 to 5.29 mg.

In this present invention, smaller amount of excipient is used for dilution and achieved desired flowability. Therefore total amount of the formulation is decreased with small amount of excipient. In addition, smaller amount of excipient provides less side effects.

In this present invention, vacuum-drum technology is used for dose forming process. In this method, composition is vacuumed and filled into separate cavities. Drums are specialized for this inhalable powder characteristics comprising tiotropium.

Example: Tiotropium bromide dry powder inhaler

| Ingredients | Amount (mg) |
|---|---|
| tiotropium bromide | 0.0217 |
| lactose monohydrate | 5.2783 |

Manufacturing Process:
Lactose monohydrate is weighted into a glass container.
The walls of shaker container are plastered with a sum of lactose monohydrate.
Lactose monohydrate and Tiotropium bromide are weighed and added into the shaker container.
A sum of lactose monohydrate is added into the shaker container and mixed for 3 minutes. This process is repeated two times more with lactose monohydrate again.
Obtained mixture is sieved through 125 μm hand sieve to obtain a homogenous mixture without causing no particle size change.
The whole mixture is taken into the same shaker container again.
The sieve is cleaned with rest amount of lactose monohydrate.
The mixture is mixed for 105 minutes with shaker container. In order to reduce electrostatic charge of the micronized active substance and to provide mixing homogenously, “electrostatic eliminator” is used during the process.

Blister Filling Process:

Vacuum-drum technology dose forming process is used for blister filling. The blister cavity is in the volume range of 15 to 19.7 mm3 and the cavity is filled preferably up to 40-60% by vacuum-drum technology dose forming process. The powder which is filled into the cavity is in the amount of 3-6 mg.

During the process parameters of vacuum-drum device are;
Vacuum pressure: −50-400 mBar
Blow pressure: 0.2-2.0 bar
In-drum cleaning: 0.5-2.0 bar
Dosing rate: 5-35 period/min
Sealing temperature: 140-200° C.
Compressive strength: 10-30 kN
Sealing time: 200-600 ms

Finally, the blister strips are coiled into the Sanohaler medical device.

1. A blister which is an integral component of a dry powder inhaler is structured from a base sheet and a lid sheet comprising at least two layers made from polymeric materials and formed or flat aluminium folio, wherein cavity that is used for storing inhalable formulation is in the volume range of 5 to 19.8 mm³ and the cavity is filled up to 25-100%.

2. The blister according to claim 1, wherein the cavity is in the volume range of 10 to 19.8 mm³, preferably in the range of 15 to 19.7 mm³.

3. The blister according to claims 1 and 2, wherein the cavity having the volume is filled up to 30-100%, preferably up to 30-70%, the more preferably up to 40-60%.

4. The blister according to claims 1 to 3, wherein the cavity that is used for storing inhalable formulation is filled about 3-6 mg of the formulation.

5. The blister according to claim 1, wherein the blister is in a strip form.

6. The blister according to claim 1, wherein the polymeric materials used for making the polymeric layers which are present in both the base sheet and lid sheet are selected from the group consisting of polyethylene, polypropylene, polystyrene, polyolefin, polyamide, polyvinyl chloride, polyurethane, vinyl acetate, polybutylmethacrylate, copolymer of vinyl chloride, polyethylene terephthalate (PET), polyvinyl chloride (PVC), ortho-phthalic aldehyde (OPA) or mixtures thereof.

7. The blister according to claim 1, wherein aluminium foil that is used in the lid sheet of the blister strip is in the thickness range of 5 to 40 μm, preferably of 10 to 30 μm.

8. The blister according to claim 1, wherein aluminium foil that is used in the base sheet of the blister strip is in the thickness range of 30 to 60 μm, preferably of 35 to 55 μm.

9. The blister according to claim 1, wherein the polymeric layer which is used in the lid sheet of the blister strip is in the thickness range of 10 to 50 μm, preferably of 15 to 35 μm.

10. The blister according to claims 6 and 9, wherein the polymeric material used for making the polymeric layer is preferably polyethylene terephthalate (PET).

11. The blister according to claim 1, wherein the polymeric layer which is used in the base sheet of the blister strip is in the thickness range of 10 to 40 μm, preferably of 15 to 35 μm.

12. The blister according to claims 6 and 11, wherein the polymeric material used for making the polymeric layer is preferably ortho-phthalic aldehyde (OPA).

13. The blister according to claim 1, further comprising another polymeric layer which is used in the base sheet of the blister strip is in the thickness range of 80 to 120 μm, preferably of 90 to 110 μm.

14. The blister according to claims 6 and 13, wherein the polymeric material used for making the polymeric layer is preferably polyvinyl chloride (PVC).

15. The blister according to any preceding claim, wherein comprising an inhalable formulation comprising tiotropium bromide and at least one pharmaceutically acceptable excipient.

16. The blister according to claim 15, wherein tiotropium bromide is present in an amount of 0.25 to 0.75% by weight of total composition, preferably it is 0.35 to 0.45% by weight of total composition.

17. The blister according to claim 15, wherein at least one pharmaceutically acceptable excipient is lactose monohydrate.

18. The blister according to claim 18, wherein lactose monohydrate is present in an amount of 98.00 to 99.99% by weight of total composition; preferably it is 99.00 to 99.99% by weight of total composition.
